(19) United States
(12) Patent Application Publication
(10) Pub. No.: US 2002/0062026 A1
(43) Pub. Date: May 23, 2002

(54) Method for preparing N-methyleneglycinates

(75) Inventors: Stefan Eils, Hanau (DE); Kai Rossen, Hanau (DE); Wilfried Jahn, Gelnhausen (DE); Ingo Klement, Pohlheim-Garbenteich (DE)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: DEGUSSA AG, Duesseldorf (DE)

(21) Appl. No.: 09/987,542

(22) Filed: Nov. 15, 2001

(30) Foreign Application Priority Data
Nov. 15, 2000 (DE) 100 56 468.2

(51) Int. Cl. C07C 229/34; C07C 229/30; C07D 213/74; C 07D 2 7/335

(52) U.S. Cl. 546/335; 548/566; 560/35; 560/168

(57) ABSTRACT

N-methyleneglycinates of formula (I)

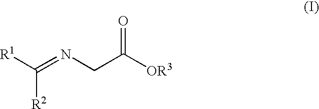

may be easily prepared in high yield by the nucleophilic substitution of α-substituted acetic esters with imines.

### BACKGROUND OF THE INVENTION

1. Field of the Invention

The present invention relates to a method for preparing N-methyleneglycinates, in particular, glycinates having the general formula (I).

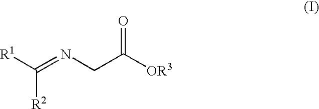

N-methyleneglycinates are important intermediates in organic chemical synthesis and are used to make biologically active substances.

2. Discussion of the Background

Several methods for preparing N-methyleneglycinates are known. For example, Ibata et al. (Chem. Lett. 1994, 1, 81-84) reacted 2-diazoacetic acid with benzophenone imine and copper catalysts. In addition, the reaction was carried out with glycine ester starting materials (Fast et al., J. Chem. Soc. Perkin Trans. 1, 1988, 3081-3084; Yaozhong et al. Synth. Commun. 1989, 19, 881-888). Glycine ester hydrochlorides and tosylates were also reacted successfully (ODonnell et al., J. Org. Chem., 1982, 47, 13, 2663-2666). It is also known that α-triflate-substituted ethyl acetate may be reacted with N-alkylated benzophenone imine (Vedejs et al, J. Org. Chem. 1987, 52, 3470-3474), and amides of acetic acid were also transformed in this way (Moenius et al., J. Labelled Compd. Radiopharm. 1999, 42, 1006-7).

### SUMMARY OF THE INVENTION

The present invention is an alternative method for preparing N-methyleneglycinates of formula (I), in which imines of formula (II) are reacted with a-substituted acetate esters of formula (III), as described below.

### DETAILED DESCRIPTION OF THE INVENTION

Methyleneglycinates having the general formula I

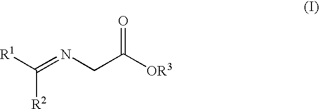

wherein R¹ and R² are independently (C₁-C₈) alkyl, (C₂-C₈) alkoxyalkyl, (C₆-C₁₈) aryl, (C₇-C₁₉) aralkyl, (C₃-C₁₈) heteroaryl, (C₄-C₁₉) heteroaralkyl, (C₁-C₈)-alkyl-(C₆-C₁₈)-aryl, (C₁-C₈)-alkyl-(C₃-C₁₈)-heteroaryl, (C₃-C₈) cycloalkyl, (C₁-C₈)-alkyl-(C₃-C₈)-cycloalkyl, (C₃-C₈)-cycloalkyl-(C₁-C₈)-alkyl, R³ is (C₁-C₈) alkyl, (C₂-C₈) alkoxyalkyl, (C₆-C₁₈) aryl, (C₇-C₁₉) aralkyl, (C₃-C₈) cycloalkyl, (C₁-C₈)-alkyl-(C₃-C₈)-cycloalkyl, may be prepared by reacting imines with the general formula II

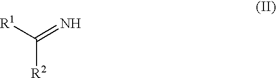

wherein R¹ and R² are as described above, with α-substituted acetate ester compounds having the general formula (III)

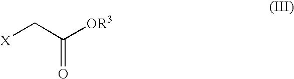

wherein R³ is as described above, and X represents a nucleofuge leaving group. The desired N-methyleneglycinate compounds may be obtained surprisingly easily, in very good yields and purity. The process of the present invention is extremely robust, and is therefore particularly suitable for use on an industrial scale. The starting materials are also relatively inexpensive and readily available, so that the process of the present invention is economically advantageous.

In principle, X may be any group well known to one skilled in the art which is suitable for this process. Such leaving groups are known in the literature (see, for example, Advanced Organic Chemistry, J. March, John Wiley and sons, 4th Ed., 1992, Chapter 10, herein incorporated by reference). X is preferably a halide, alkyl- or arylsulfonate, or carboxylate, particularly chloride, bromide, triflate, mesylate, p-tosylate, trifluorocarboxylate, p-nitrobenzoate, or acetate, etc.

The (C₁-C₈) alkyl groups may be, for example, methyl, ethyl, n-propyl, isopropyl, n-butyl, isobutyl, sec-butyl, tent-butyl, pentyl, hexyl, heptyl, or octyl, including all positional isomers thereof. The (C₁-C₈) alkoxy group may be, for example, (C₁-C₈) alkyl groups as described above, bonded to the structures of formula (I) or (II) through an oxygen atom. The (C₂-C₈) alkoxyalkyl groups are groups in which the alkyl chain is interrupted by at least one oxygen atom, wherein no two oxygen atoms may be bonded to one another. The number of carbon atoms indicates the total number of carbon atoms contained in the group (e.g., —CH₂—O—CH₃).

The groups described above may be also substituted one or more times with halogen atoms, and/or groups containing N, O, P, S, or Si atoms. For example, such groups may include alkyl groups of the type mentioned above that have one or more of these hetero atoms in their chain or that are bonded to the molecule through one of these hetero atoms.

The (C₃-C₈) cycloalkyl groups may be, for example, cyclopropyl, cyclobutyl, cyclopentyl, cyclohexyl, or cycloheptyl groups, etc. These cycloalkyl groups may also be substituted with one or more halogen atoms and/or groups containing N, O, P, S, or Si atoms and/or may have N, O, P, or S atoms in the ring, for example, groups such as 1-, 2-, 3-, or 4-piperidyl, 1-, 2-, or 3-pyrrolidinyl, 2- or 3-tetrahydrofuryl, or 2-, 3-, or 4-morpholinyl groups.

A (C₃-C₈)-cycloalkyl-(C₁-C₈)-alkyl group is a cycloalkyl group as discussed above that is bonded to the molecule through an alkyl group as discussed above.

In the context of the present invention, (C₁-C₈) acyloxy is an alkyl group, as defined above, with a maximum of 8 carbon atoms, that is bonded to the molecule through a COO— function.

A (C₆-C₁₈) aryl group is an aromatic group with 6 to 18 carbon atoms, particularly including, for example, phenyl, naphthyl, anthryl, phenanthryl, and biphenyl groups, or such aromatic groups substituted with (C₁-C₈) alkyl, (C₁-C₈) alkoxy, (C₁-C₈) acyl, (C₁-C₈) acyloxy, NO₂, NR¹R², SR¹, SH, SOR¹, SO₂R¹, or halogen.

A (C₇-C₁₉) aralkyl group is a (C₁-C₈) alkyl group bonded to the structures of formula (I) or (II) through a (C₆-C₁₈) aryl group.

A (C₃-C₁₈) heteroaryl group, in the context of the present invention, is a five-, six-, or seven-membered aromatic ring system consisting of 3 to 18 carbon atoms that has hetero atoms such as nitrogen, oxygen, or sulfur in the ring. In particular, such heteroaromatic groups may be, for example, 1-, 2-, or 3-furyl, 1-, 2-, or 3-pyrrolyl, 1-, 2-, or 3-thienyl, 2-, 3-, or 4-pyridyl, 2-, 3,-, 4-, 5-, 6-, or 7-indolyl, 3-, 4-, or 5-pyrazolyl, 2-, 4-, or 5-imidazolyl, acridinyl, quinolinyl, phenanthridinyl, and 2-, 4-, 5-, or 6-pyrimidinyl.

A (C₄-C₁₉) heteroaralkyl group may be a heteroaromatic system corresponding to the (C₇-C₁₉) aralkyl group described above, in which some of the aromatic carbon atoms are substituted with hetero atoms such as nitrogen, oxygen, or sulfur in the aromatic ring.

Suitable halogen atoms include, for example, fluorine, chlorine, bromine, and iodine.

The reaction of the present invention is preferably carried out in the presence of a base. Any organic or inorganic base familiar to one skilled in the art may be used. The base is preferably a trialkylamine such as triethylamine or diisopropylethylamine, or a carbonate such as sodium carbonate or sodium bicarbonate.

Any organic solvent recognized by one skilled in the art for the reaction of the present invention may be used, provided it is inert toward substitution. A polar, aprotic solvent is preferred. Especially preferred solvents are acetonitrile, N-methylpyrrolidone, dimethyl sulfoxide, or dimethylformamide.

The reaction temperature should be chosen so that the rate of reaction is suitably high, but also minimizes the formation of byproducts as much as possible. Preferably, the reaction temperature is between 20° C. and 150° C., more preferably between 60° C. and 90° C.

The preferred procedure according to the present invention is to dissolve the α-X-substituted acetate ester in the solvent of choice, and then to add the imine. The base is subsequently added to the reaction mixture and heat is applied with stirring. In order to work up (i.e., recover) the product, the reaction mixture is cooled and then neutralized, preferably with a carboxylic acid such as acetic acid, for example, and then water is added. The mixture is then cooled until a precipitate forms. The precipitate is then filtered off, washed, and dried. This method for preparing these relatively complicated imine structures is extremely attractive as an industrial process, because the starting materials may be recycled very readily and used again for the reaction. The present method for preparing N-methyleneglycinates is therefore advantageous from both an economic and ecological perspective.

### EXAMPLES

Having generally described this invention, a further understanding can be obtained by reference to certain specific examples which are provided herein for purposes of illustration only, and are not intended to be limiting unless otherwise specified.

### Example 1

3.90 g of tert-butyl 2-bromoacetate was dissolved in 20 ml of acetonitrile, and 3.62 g of benzophenone imine and 2.58 g of diisopropylethylamine were added. The solution was heated under reflux with stirring for 8 h. The reaction solution was then neutralized with 50% aqueous acetic acid at room temperature, and an additional 30 ml of water was added. Upon cooling in an ice bath, the product precipitated out and was filtered off by suction through a sintered glass funnel. The precipitate was washed with a small amount of cold 90% ethanol, dried on a filter by suction, and dried over phosphorus pentoxide under vacuum. In this way 4.45 g (75% yield) of tert-butyl N(diphenylmethylene)glycinate was obtained (m.p. 114.5° C.).

### Example 2

3.62 g of tert-butyl 2-chloroacetate was dissolved in 20 ml of N-methylpyrrolidinone, and 0.6 g of potassium iodide, 3.32 g of potassium carbonate, and 3.61 g of benzophenone imine were added. The reaction mixture was stirred for 6 h at 80° C. and was then cooled to room temperature. 20 ml each of ethanol and water were added. The basic solution was brought to pH 5.5 by adding 8 ml of acetic acid. After adding an additional 5 ml of ethanol and 30 ml of water, the product precipitated out when cooled in an ice bath. It was filtered off through a sintered glass funnel by suction, washed with 70% ethanol, and dried under vacuum. In this way, 4.0 g (68% yield) of tert-butyl N-(diphenylmethylene)glycinate was obtained.

The priority document of the present application, German application DE 100 56 468.2, filed Nov. 15, 2000, is incorporated herein by reference.

Obviously, numerous modifications and variations on the present invention are possible in light of the above teachings. It is therefore to be understood that within the scope of the appended claims, the invention may be practiced otherwise than as specifically described herein.

What is claimed as new and is intended to be secured by Letters Patent is:

1. A method of preparing a N-methyleneglycinate of formula (I), 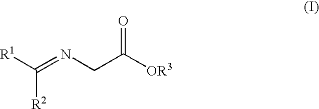
comprising:
reacting an imine of formula (II): 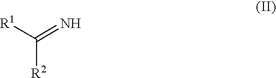
with a compound of formula (III): 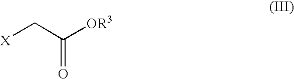
wherein R¹ and R² are independently selected from the group consisting of a (C₁-C₈) alkyl group, a (C₂-C₈) alkoxyalkyl group, a (C₆-C₁₈) aryl group, a (C₇-C₉) aralkyl group, a (C₃-C₁₈) heteroaryl group, a (C₄-C₁₉) heteroaralkyl group, a (C₁-C₈)-alkyl-(C₆-C₁₈)-aryl group, a (C₁-C₈)-alkyl-(C₃-C₁₈)-heteroaryl group, a (C₃-C₈) cycloalkyl group, a (C₁-C₈)-alkyl-(C₃-C₈)-cycloalkyl group, and a (C₃-C₈)-cycloalkyl-(C₁-C₈)-alkyl group; R³ is selected from the group consisting of a (C₁-C₈) alkyl group, a (C₂-C₈) alkoxyalkyl group, a (C₆-C₁₈) aryl group, a (C₇-C₁₉) aralkyl group, a (C₃-C₈) cycloalkyl group, and a (C₁-C₈)-alkyl-(C₃-C₈)-cycloalkyl group; and X is a nucleofuge leaving group.

2. The method of claim 1, wherein X is selected from the group consisting of a halide, an alkyl-sulfonate group, an arylsulfonate group, and a carboxylate group.

3. The method of claim 2, wherein X is selected from the group consisting of chloride, bromide, triflate, mesylate, p-tosylate, trifluorocarboxylate, p-nitrobenzoate, and acetate.

4. The method of claim 1, wherein the reaction is carried out in the presence of a base.

5. The method of claim 2, wherein the reaction is carried out in the presence of a base.

6. The method of claim 4, wherein the base is selected from the group consisting of a trialkylamine and a carbonate.

7. The method of claim 6, wherein the trialkylamine is triethylamine or diisopropylethylamine.

8. The method of claim 6, wherein the carbonate is sodium carbonate or sodium bicarbonate.

9. The method of claim 1, wherein the reaction is carried out in an inert solvent.

10. The method of claim 2, wherein the reaction is carried out in an inert solvent.

11. The method of claim 9, wherein the inert solvent comprises a polar organic solvent.

12. The method of claim 11, wherein the polar organic solvent is selected from the group consisting of acetonitrile, N-methylpyrrolidone, dimethyl sulfoxide, and dimethylformamide.

13. The method of claim 1, wherein the reaction is carried out at a temperature in the range of 20° C. to 150° C.

14. The method of claim 2, wherein the reaction is carried out at a temperature in the range of 20° C. to 150° C.

15. The method of claim 1, wherein the reaction is carried out at a temperature in the range of 60° C. to 90° C.

16. The method of claim 1, further comprising:
neutralizing a mixture comprising a N-methyleneglycinate of formula (I), formed by reacting the imine of formula (II) and the compound of formula (III), with a carboxylic acid.

17. The method of claim 1, further comprising precipitating said N-methyleneglycinate of formula (I) by cooling, after said reacting.

18. The method of claim 1, further comprising:
neutralizing a mixture comprising a N-methyleneglycinate of formula (I), formed by reacting the imine of formula (II) and the compound of formula (III), with a carboxylic acid; then
precipitating said N-methyleneglycinate of formula (I) by cooling.
